(19) United States
(12) Patent Application Publication
(10) Pub. No.: US 2012/0315289 A1
(43) Pub. Date: Dec. 13, 2012

(54) Non-polysaccharide constituent of genus Dendrobium, usage for the non-polysaccharide constituent of genus Dendrobium and extracting method thereof

(76) Inventors: Hsin-Sheng Tsay, Taichung City (TW); Chang-Chi Hsieh, Taichung City (TW); Chin-Tung Wu, Taichung City (TW); Yu-Chang Chen, Taichung (TW); Chao-Lin Kuo, Taichung (TW); Shu-Fang Lo, Chiayi City (TW)

(21) Appl. No.: 13/281,813

(22) Filed: Oct. 26, 2011

(30) Foreign Application Priority Data
Jun. 13, 2011 (TW) 100120579

(51) Int. Cl. A61K 31/00; A61P 37/08; C07G 99/00

(52) U.S. Cl. 424/184.1; 568/300

(57) ABSTRACT

The present invention discloses a non-polysaccharide constituent for positively regulating the immune system in the therapy of allergic diseases. The non-polysaccharide constituent is extracted from genus Dendrobium by incubating in alcohol and extracting using solvents with different polarity.

### CLAIMED TECHNOLOGY FIELD

The present invention relates to a bioactive extract and the extracting method, and especially a non-polysaccharide constituent of genus Dendrobium and extracting method thereof. The non-polysaccharide constituent could be applied to efficiently cure or suppress the occurrence of allergic diseases.

### PREVIOUS TECHNOLOGY

(Introduction)

Genus Dendrobium, a member of family Orchidaceae, distributing in tropical and subtropical area is usually applied as cut flowers and potted orchids for ornament. Including 15 species found in Taiwan, there are 1500 species in genus Dendrobium in the world. Either dry or fresh stem of genus Dendrobium is a precious Chinese herb named as Dendrobium spp. (Herbs Dendrobii) that is widely used in traditional therapies for bringing down a fever, ophthalmology diseases or nourishing purpose.

According to the previous studies, genus Dendrobium includes many substances, such as phenanthrenes, bibenzyls, fluorenones, sesquiterpenes and alkaloids, containing various bioactivities and medical applications. Recent studies also demonstrated the anti-inflammatory, antioxidant and anti-allergic activities in these compounds. Dendrobium tosaense MAKINO is one of the medical genus Dendrobium plant applied as functional health food in Taiwan. So far, it is feasible to largely culture the Dendrobium tosaense, which contains qurecetin for removing free radical, in the warm room by plant tissue culture. Furthermore, quercetin (3,5,7,3′,4′-pentahydroxyflavone) is a kind of flavone which widely exists in the vegetables and fruits. It is quite beneficial for human health due to the anti-oxidation activity. The chemical structure of the queretin is aglycones, which belongs to non-polysaccharide polyphenols.

Atopic dermatitis (AD) is an allergic disease, preferentially occurring in babyhood with unknown reason, with obvious features such as itch, eczematous lesions, xerosis and lichenification. In addition, this allergic disease also associates with other allergic diseases such as asthma, allergic rhinitis, urticaria and food allergy that are the general problems for human health in the world. The incidence of atopic dermatitis is about 10-20% in childhood with a growing trend. The causing mechanisms of atopic dermatitis include genetic factors, environment, skin barrier disorders and immune response that are under investigation.

Mast cell is the key effector in IgE inducing allergic diseases that is activated by the interaction between IgE and high-affinity IgE receptor, FcεRI, on the cell surface. After the activation, mast cell undergoes degranulation to secrete the bioactive substances such as platelet activating factor (PAF), histamine, leukotriene C4 and prostaglandin E2 (PGE2), which play important role in allergy. Atopic dermatitis is usually related to IgE mediated allergic mechanism induced by dust mite, ovalbumin, seafood and fungus. Most patients of atopic dermatitis bear excessive expression of cytokine and production of IgE. Among these cytokines aberrantly produced in atopic dermatitis patients, interleukin-4 (IL-4), interleukin-5 (IL-5) and interleukin-3 (IL-3) are produced by type-2 T helper cells (T_{H}2 cells). In addition to the increase of T_{H}2 cells and cytokines produced by T_{H}2 cells, the production of IFN-γ and interleukin-12 (IL-12) are decreased in these patients. These studies suggest that the occurrence of atopic dermatitis is related to the differentiation of T helper cells. Thus, it is reasonable to cure atopic dermatitis by regulation of T_{H}1/T_{H}2 balance through suppressing T_{H}2 cells related factors and enhancing T_{H}1 cells related factors.

### CONTENT OF INVENTION

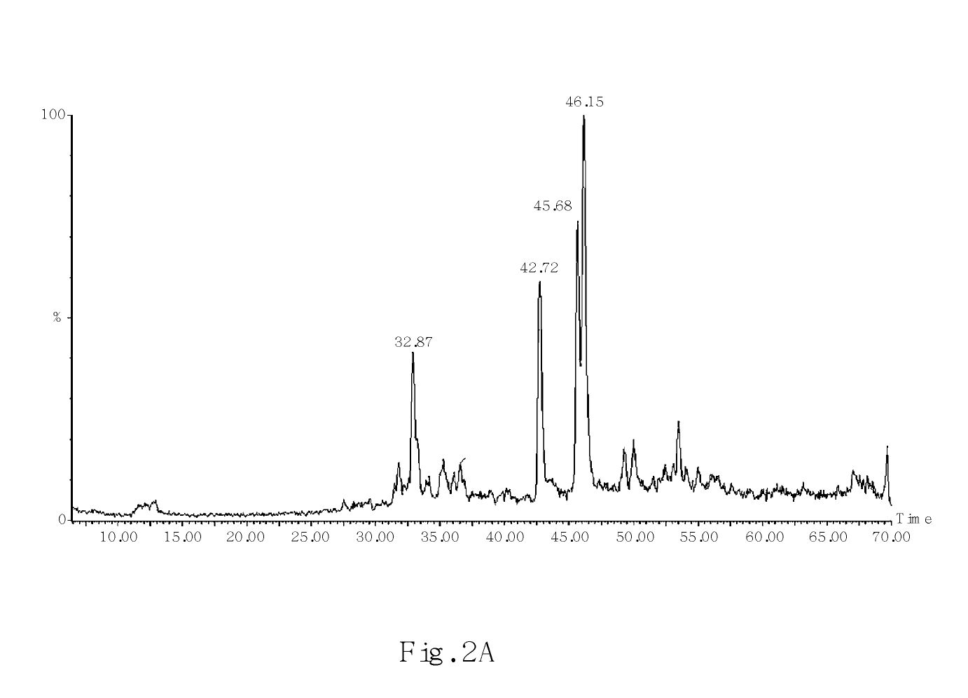

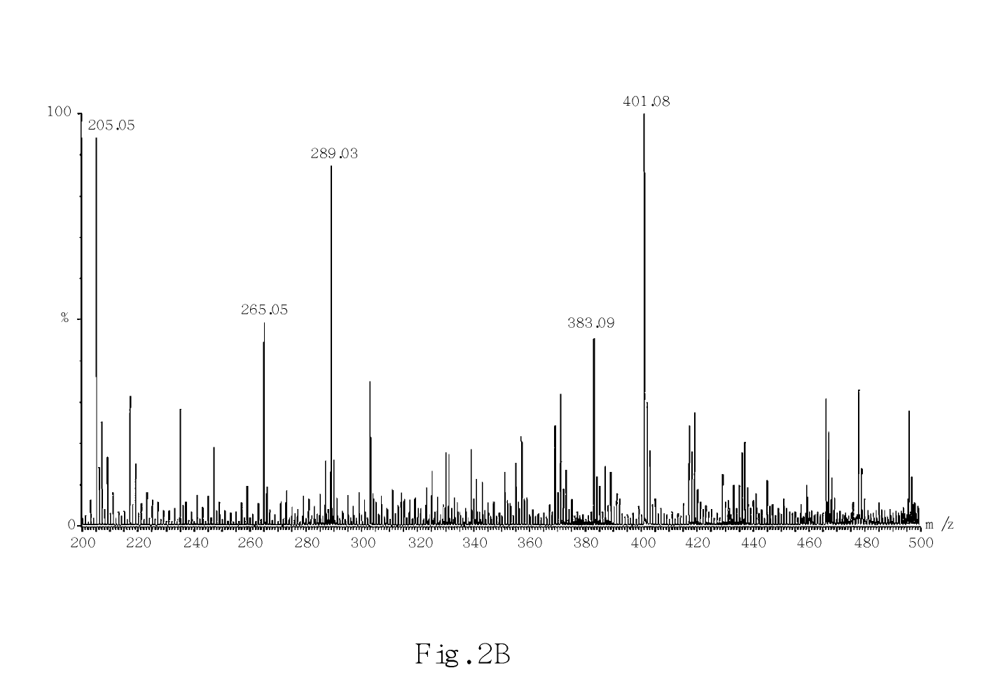

Base of the foregoing, one aspect of the invention is to provide a non-polysaccharide constituent extracted from genus Dendrobium plant, shown in FIG. 2A, as the effective component for the therapy of allergic disease, wherein the allergic disease is correlated with the increase of IgE, cytokines expression and T_{H}2 cells.

Another aspect of the invention is to provide a non-polysaccharide constituent of genus Dendrobium plant for positive regulation of the immune system in allergic disease patients.

In another aspect of the invention also provides a method of extracting a non-polysaccharide constituent from genus Dendrobium plant through the following steps:

Step A: taking a specific part of a genus Dendrobium plant.

Step B: incubating the specific part of genus Dendrobium taken in step A in an alcohol to obtain a first product.

Step C: extracting a non-polysaccharide constituent from the first product by using at least one low-polarity solvent.

Furthermore the step-A is to take the stem of dry genus Dendrobium plant; the alcohol of the step-B is methanol; and the step-C extracts the first product of step-B by two solvents with different polarity, including the following steps:
C′: extracting a second product by using n-hexane.
C″: obtaining a non-polysaccharides constituent of Dendrobium tosaense from the product in C′ by using ethyl acetate (EA).

The non-polysaccharide constituent extracted by the provided method, shown in FIG. 2A, having the effect for positively regulating allergic diseases. These allergic disease which include atopic dermatitis, asthma and urticarial, are caused by the abnormal increase of IgE, cytokine and T_{H}2 cells.

### MATERIAL AND METHODS

The present invention discloses a non-polysaccharide constituent extracted from a genus Dendrobium plant as the effective component in the therapy of allergic diseases through positively regulating immune system in the patients. These allergic diseases, including atopic dermatitis, asthma and urticarial, are correlated with the increase of IgE, cytokine and T_{H}2 cells. The present invention also provides a method of extracting the non-polysaccharide constituent from the stem of the genus Dendrobium plant, wherein the genus Dendrobium plant will be illustrated by Dendrobium tosaense in the examples mentioned below.

A fresh Dendrobium tosaense is dehydrated at room temperature or low-temperature and followed by grind for obtaining the powder for the further extraction with better quantitation purpose. The powder of Dendrobium tosaense is dissolved in the methanol to further extract the non-polysaccharide constituent by using two solvents with different polarity. The components in the non-polysaccharide constituent of Dendrobium tosaense, shown in FIG. 2A, has the positive effect for regulating immune system.

The present invention will further be illustrated by variable examples with reference made to the Figures, wherein:

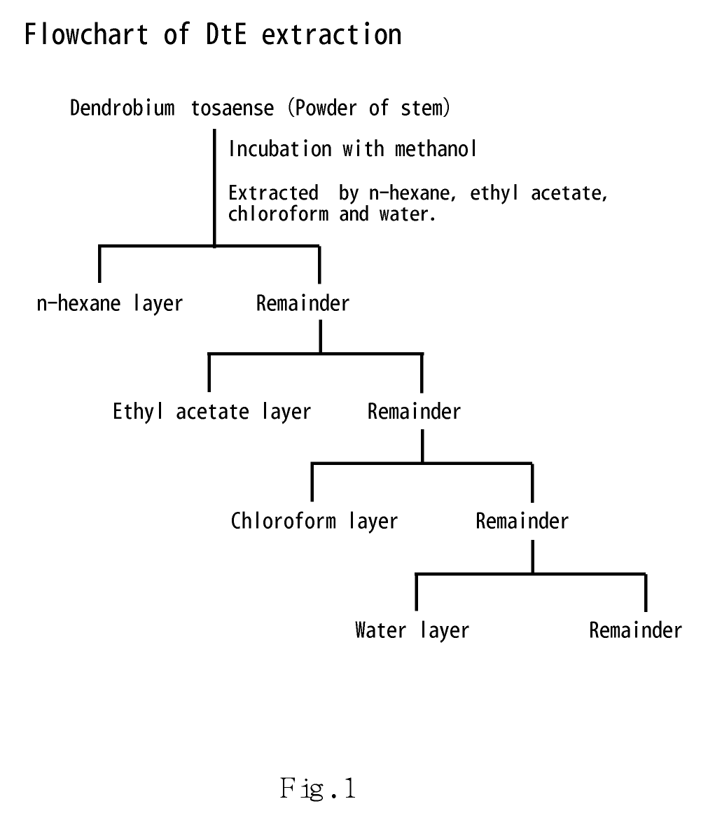

FIG. 1 illustrates the extraction process for obtaining the non-polysaccharide constituent from Dendrobium tosaense.

FIG. 2A is the spectrum of the non-polysaccharide constituent of Dendrobium tosaense analyzing by HPLC.

FIG. 2B is the spectrum of the non-polysaccharide constituent of Dendrobium tosaense analyzing by mass spectrometry (MS).

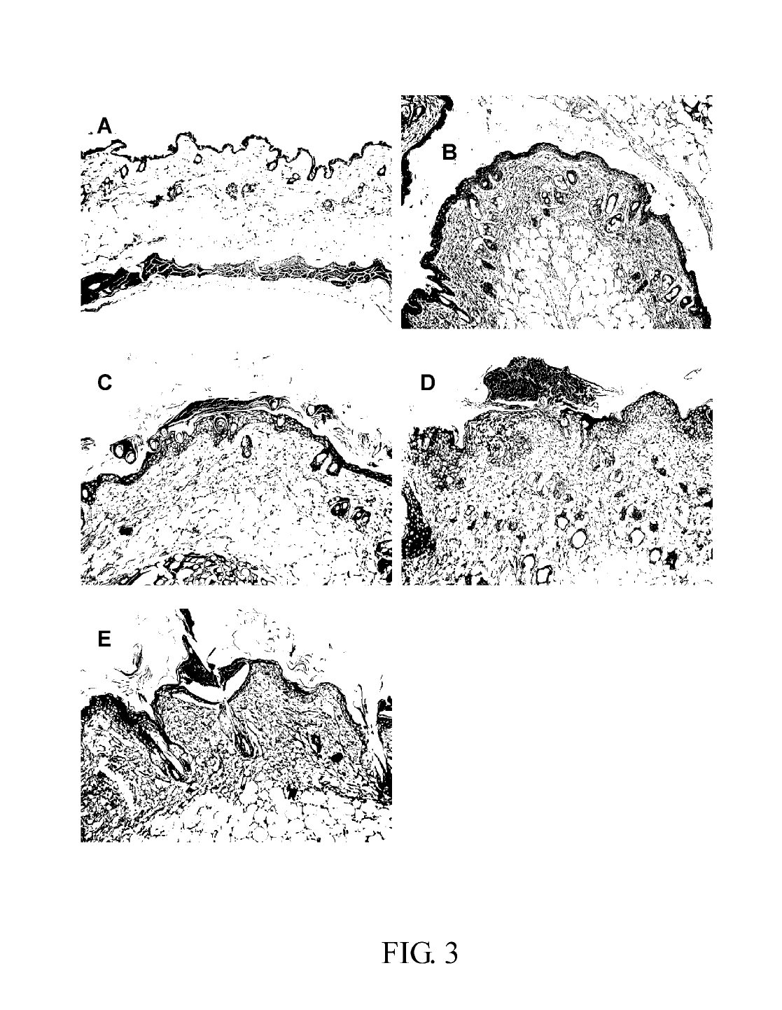

FIG. 3 shows the histology of mouse skin by H&E staining.

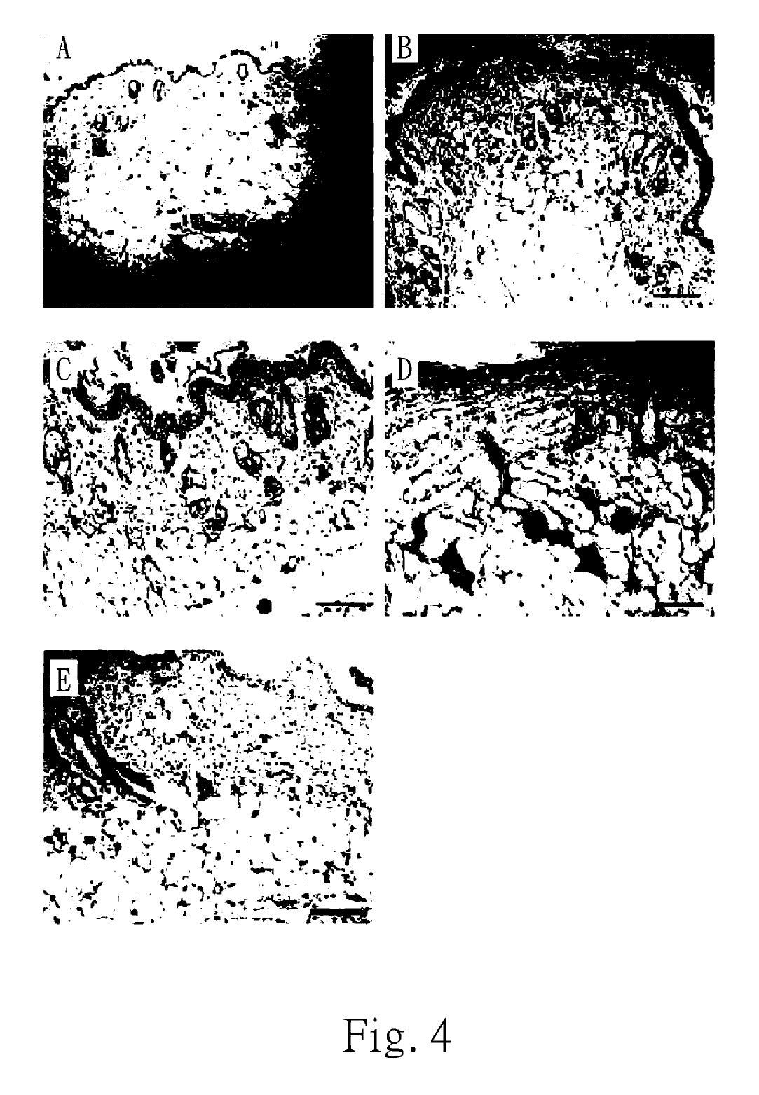

FIG. 4 shows the mast cells in mouse skin by toluidine blue staining.

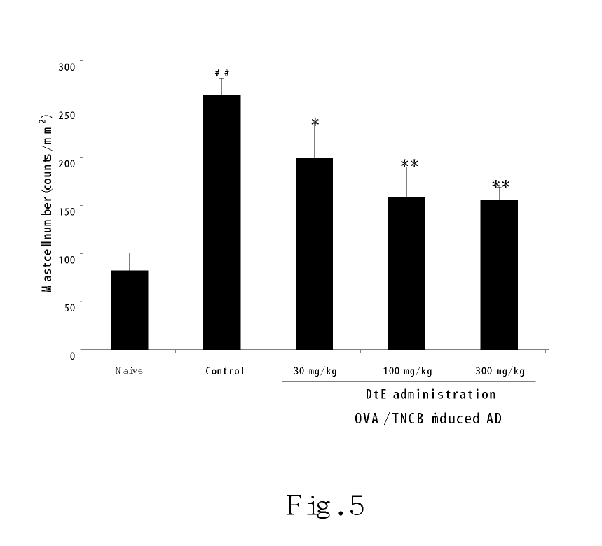

FIG. 5 is the bar chat of the altered cell number of mast cells in the mice treating with different administrations.

### EXAMPLE 1

### Material

The Dendrobium tosaense used in this extraction, identified by China Medical University and preserved in the Herbarium, College of Pharmacy, China Medical University (Sample ID: CMCDT0303), is cultured in the warm room by plant tissue culture. In addition, the internal transcribed spacer of Dendrobium tosaense genome (genebank registration number: HM590367) is used to identify the genotype.

### EXAMPLE 2

### Extracting the Non-Polysaccharide Constituent

According to FIG. 1, the dehydrated stem of Dendrobium tosaense of the example 1 is taken for grinding into powder. 30 g powder ground from the stem of Dendrobium tosaense is dissolved in 300 uL methanol and followed by sonication for 10 minutes. The methanol-extracted substances are sequentially separated by n-hexane, ethyl acetate (EA), chloroform and water. All fractions are filtered for decompressing evaporation to obtain the remainder for further the analysis by LC/MS. The LC/MS analysis demonstrates that the solution extracted from ethyl acetate contains the richest non-polysaccharide constituent composed of the components as shown in FIG. 2. FIG. 2A shows that the non-polysaccharide constituent contains four components with the retention time at 32.87, 42.72, 45.68 and 46.15 minutes. Furthermore, the molecular weight of this non-polysaccharide constituent is 302 Daltons and its chemical fomula is C₁₅H₁₀O₇, wherein FIG. 2A shows the spectrum of the constituent analyzed by HPLC, and FIG. 2B represents the spectrum analyzed by LC/MS with the product sorted by HPLC.

Thus, the non-polysaccharide constituent (stand for DtE in the further description) extracted by using ethyl acetate will be provided for the further examples.

### EXAMPLE 3

### Generating the Mouse Model of Allergy

BALB/c female mice with body weight 18˜22 gram are purchased from National Laboratory Animal Center. These mice are maintained in specific conditions including consistent temperature between 21° C. to 24° C., regular light cycle (12 hours for both light and dark phase), autoclaved feedstuff and distilled water for two weeks. These mice are used to generate the mouse model of allergy when they are 8 weeks old.

The mouse model of allergy is generated according to the previous study (Dia et al., 2002). 7% of 2,4,6-tintrochlorobenzene (TNCB) is dissolved in the acetone and olive oil mixture (4:1) and followed by adding 25 μg ovalbumin. This solution is applied at the local region of back skin to harvest the mice with allergic disease on the skin, such as atopic dermatitis, for the further experiments after 7 days.

### EXAMPLE 4

### Animal Experiment

The mouse model of allergy generated in example 3 is treated with DtE at 30 mg/kg, 100 mg/kg and 300 mg/kg dissolved in carboxymethyl cellulose (CMC) by oral feeding. The experimental group includes the allergic mice fed with sterile carboxymethyl cellulose at 10 ml/kg. In addition, the naive group includes the normal mice or allergic mice without DtE administration. The blood sample is collected for the immune or blood analysis on seventh day after DtE administration. In addition, the skin tissue is taken from the sacrificed mice as sample with fixation in 10% formalin for the further histopathology examination. Furthermore, the spleen of the sacrificed mice is taken for the culture of splenocyte with activation by mitogen (ConA, 5 μm/ml) for 24 to 72 hours. The supernatant of the culture is collected for analyzing the production of IL-4, IL-6 and IFN-γ by sandwich enzyme-linked immunosorbent assay (sandwich ELISA).

### EXAMPLE 5

### Observing the Mouse Cutaneous Histology by H&E Staining

The paraffin embedded mouse skin samples are sectioned with appropriated thickness of 5 μm. After rehydration, the sections are stained with hematoxylin and washed by rinse in water for five minutes. Subsequently, excessive hematoxylin is removed by acid alcohol that contains 1% hydrochloric acid for 3 to 5 seconds, and followed by washing with water. The sections are further stained with eosin for five minutes and washed by flowing water until the nuclei turn blue. Sequentially dehydration of the sections is performed with 70%, 80%, 90% and 100% ethanol and followed by mounting for the histopathology observation. As the data shown in FIG. 3, the histology of normal mice and unfed allergic mice are present in FIGS. 3A and 3B, respectively. In addition, the histology of allergic mice fed with 30 mg/kg, 100 mg/kg and 300 mg/kg DtE are shown in FIG. 3C to 3E.

According to FIGS. 3A and 3B, the thicker epidermis and infiltrating leukocytes in the dermis are revealed in the unfed allergic mice with the comparison of normal mice. Interestingly, DtE administration in the allergic mice improves these abnormalities of skin from moderate to miner, which include the keratosis of epidermis and monocytes infiltration in dermis. Furthermore, higher dose of DtE fed to the allergic mice, better effect is observed in these mice (FIG. 3C to 3E). This result suggests that DtE contains the potency to improve the atopic dermatitis with a dose dependent manner.

### EXAMPLE 6

### Observing Mast Cells by Using Toluidine Blue Staining

Because mast cell is the critical effector in the atopic dermatitis, master cell infiltration is further observed in the mouse skin. Deparaffinization, rehydration and staining with toluidine blue reagent (1% toluidine blue and 1% potassium aluminum sulfate) for 2˜3 minutes are performed with the descripted sections. After wash with distilled water and dehydration with ethanol, the histology is observed using the microscope and shown in FIG. 4 and FIG. 5. In FIG. 4, the histology of normal mice and unfed allergic mice are shown in FIGS. 4A and 4B. In addition, the histology of the allergic mice fed with 30 mg/kg, 100 mg/kg and 300 mg/kg DtE are shown in FIG. 4C˜4E. Furthermore, the cell number of mast cells in the area unit of skin is further calculated and shown in FIG. 5. According to FIG. 4 and FIG. 5, the results suggest that the infiltrating mast cell is increased in unfed allergic mice but obviously reduced with DtE administration.

### EXAMPLE 7

### Analyzing the Amount of IgE in the Allergic Mice Using ELISA

The sera with different dilutions (1:20 to 1:100 dilution) are loaded into the 96-wells plate coated with ovalbumin-specific antibodies (OVA) for incubation at 4° C. overnight. The 96-wells plate is washed and followed by the incubation with HRP-conjugated goat anti-mouse IgE and IgG1 secondary antibody for one hour. After the incubation, the plate is washed and followed by color reaction by SureBlue Reserve TMB Microwell Peroxidase Substrate. Result is measured by spectrophotometer at 450 nm, and shown in table 1.

TABLE 1
The amount of IgE and IgG1 in the sera collected from the
allergic mice with administration.

| | Anti-OVA antibodies | |
|---|---|---|
| Treatment | Immunoglobulin G1 | Immunoglobulin E |
| Normal mice | 0.16 ± 0.02 | 0.31 ± 0.01 |

Mouse model of allergy

| | | |
|---|---|---|
| Unfed | 0.56 ± 0.10^{###} | 0.45 ± 0.04^{###} |
| Fed with DtE 30 mg/kg | 0.36 ± 0.03** | 0.34 ± 0.02** |
| Fed with DtE 100 mg/kg | 0.43 ± 0.04 | 0.32 ± 0.02** |
| Fed with DtE, 300 mg/kg | 0.43 ± 0.03 | 0.32 ± 0.02*** |

Table 1 shows that the amounts of IgG1 and IgE produced in unfed allergic mice are obviously increased as compared with normal mice. With the administration of 30 mg/kg, 100 mg/kg and 300 mg/kg DtE for one week, the amounts of IgG1 and IgE in the sera are obviously reduced in the allergic mice. These results indicate that DtE plays important role in positively regulating the mechanism of anti-allergy.

### EXAMPLE 8

### Examining the Cytokine Production in the Mice

The spleen taken from the sacrificed mice in example 4 is cultured with activation by ConA for 24˜72 hours. Following the activation, the supernatant is collected for the sandwich ELISA analysis to measure the production of cytokines such as IL-4, IL-6 and IFN-γ. In detail, the capture antibodies are incubated in the plate at 4° C. overnight and followed by washing with 0.05% Tween20 containing Dulbecco's modified phosphate buffered saline at room temperature for 60 minutes. Samples and standards are added into the wells and incubated at room temperature for 2 hours, and followed by wash to remove the non-specific binding. The detecting antibodies are added into the wells and incubated for 1 hour, and followed by washing. After the incubation with substrate solution TMB in dark, the enzyme activity is terminated by 2N sulfuric acid. The absorption is detected by spectrophotometer and shown in table 2.

TABLE 2
Altered cytokines production in the allergic mice with different
DtE administration.

| Administration | IL-4, pg/ml | IFN-γ, ng/ml | IL-6, pg/ml |
|---|---|---|---|
| Normal | 12.55 ± 8.38 | 3.10 ± 1.27 | 478.1 ± 766 |

Allergic model mice

| | | | |
|---|---|---|---|
| Unfed | 33.23 ± 15.53 | 1.13 ± 0.50^{##} | 445.3 ± 78.444 |
| Feed with DtE | 11.30 ± 7.52 | 2.43 ± 0.85* | 584.0 ± 74.959 |
| 30 mg/kg | | | |
| Feed with DtE | 6.61 ± 5.31* | 2.41 ± 0.60* | 516.0 ± 121.748 |
| 100 mg/kg | | | |
| Feed with DtE | 4.40 ± 2.93* | 2.60 ± 0.99* | 391.3 ± 73.694 |
| 300 mg/kg | | | |

From table 2, the production of IL-4 is obviously reduced in the allergic mice after DtE administration by comparing with unfed allergic mice. In contrast, the production of IFN-γ is obviously increased in the allergic mice fed with DtE. These results suggest that DtE administration provides the environment for negatively regulating T_{H}2 cells and enhancing T_{H}1 cells.

### EXAMPLE 9

### Analyzing Leukocyte Populations in Spleen and T Cell Subsets in Lymph Node Nearby the Wounded Skin

The leukocytes in spleen and T cells in the lymph node nearby the wounded area are analyzed according to the cell type-specific antigens on the surface with fluorescence-conjugated monoclonal antibodies. 50 μl suspension containing 5×10⁵ cells is taken for incubation with saturated concentration of fluorescein, phycoerythrin (PE) or PE-Cy5 conjugated monoclonal antibodies in the dark on ice for minutes. The stained cells are washed with 1% sodium azide containing DPBS buffer after the incubation. The cells are centrifuged at 300×g, 4° C. for 10 minutes and re-suspended in 200 μl analyzing buffer that is composed of 2% fetal bovine serum and 0.1% sodium azide in the DPBS. The monoclonal antibodies used in the analysis are listed in table 3.

TABLE 3

| Lymphocyte | Monoclonal antibodies |
|---|---|
| B cell | CD19, CD45 |
| T cell | CD3, CD45 |
| T cytotoxic cell | CD8, CD3 |
| Treg cel | CD25, CD4 |
| Th cells | CD3, CD4 |
| Th1 cells | Tim-3, CD4 |
| Th2 cells | CD278, CD4 |

Examination of the antigen expression on splenocyte in the spleen and lymphocyte in the lymph node nearby the wounded area in skin using flow cytometry. These results are summarized in table 4 and table 5

TABLE 4
Analyzing the splenocyte population

| | B cell | Tc cells | Th cells | |
|---|---|---|---|---|
| Adminis- | (CD19+, | (CD8+, | (CD4+, | Treg cells |
| tration | CD45+) | CD3+) | CD3+) | (CD25+, CD4+) |
| Normal | 58.40 ± 1.31 | 16.11 ± 0.99 | 18.95 ± 0.68 | 4.77 ± 0.18 |
| mice | | | | |

Allergic mice model

| | | | | |
|---|---|---|---|---|
| Unfed | 60.85 ± 0.98 | 15.47 ± 1.04 | 18.05 ± 1.32 | 3.66 ± 0.33^{##} |
| Feed with | 60.63 ± 2.14 | 16.59 ± 0.60 | 18.33 ± 0.45 | 4.69 ± 0.24** |
| DtE 30 | | | | |
| mg/kg | | | | |
| Feed with | 61.29 ± 2.02 | 15.16 ± 0.48 | 18.87 ± 1.59 | 4.62 ± 0.12** |
| DtE 100 | | | | |
| mg/kg | | | | |
| Feed with | 61.20 ± 1.80 | 16.49 ± 1.39 | 18.53 ± 0.80 | 4.68 ± 0.26** |
| DtE 300 | | | | |
| mg/kg | | | | |

TABLE 5
Analyzing the lymphocyte populations in the lymph
node nearby the wounded area in skin

| | Th cells | | |
|---|---|---|---|
| | (CD4+, | Th1 cells | Th2 cells |
| Administration | CD3+) | (Tim-3+, CD4+) | (CD278+, CD4+) |
| Normal mice | 47.76 ± 2.12 | 8.47 ± 0.29 | 3.09 ± 0.12 |

Allergic mice model

| | | | |
|---|---|---|---|
| Unfed | 45.26 ± 0.88 | 6.57 ± 0.20^{###} | 5.27 ± 0.33^{###} |
| Feed with DtE | 41.83 ± 1.92 | 7.21 ± 0.21 | 4.21 ± 0.27** |
| 30 mg/kg | | | |
| Feed with DtE | 41.11 ± 1.41* | 6.75 ± 0.26 | 4.33 ± 0.24** |
| 100 mg/kg | | | |
| Feed with DtE | 40.99 ± 0.45* | 6.91 ± 0.15 | 4.30 ± 0.19** |
| 300 mg/kg | | | |

Table 4 shows the obvious reduction in cell number of Treg cells in spleen of the unfed allergic mice. In addition, although the cell number of B cells, T_{H} cells and T_{c} cells are similar with unfed allergic mice, the cell number of Treg cells is resorted by DtE administration. These results suggest that DtE administration participates in suppression of allergic dermatoid through enhancing the immune-suppression. Furthermore, the T_{H}2 cell is obviously reduced in the lymph node nearby the wounded area in the allergic mice with DtE administration. This result means that the polarization of T_{H}2 cell plays critical role in local immune-regulation.

According to these results, the non-polysaccharide constituent extracted from genus Dendrobium could obviously suppresses the epidermal damage and mast cell degranulation, up-regulate IFN-γ production, suppress IL-4 production and inhibit the excessive production of IgE or IgG1. In detail, DtE administration creats the suppressive environment for the activation of T_{H}2 cell through reducing the expression of IL4 and enhancing the expression of T_{H}1 cell produced IFN-γ. The activation of T_{H}2 cell is achieved by obviously increasing the palarization of T_{H}2 and anti-ovalbumin IgE. In addition, the obvious increase of Treg cell in the spleen maintains the immunosuppression to obviously reduce the differentiated T_{H}2 cell.

In summary, the non-polysaccharide constituent actually contains the pharmacological activity for positively regulating the allergic disease through control of the T_{H}1/T_{H}2 balance to reduce IgE production.

The above-mentioned specification is only for detailedly describing the examples of the invention and shall not be construed as a limitation of the scope of the invention Thus, t any modification or change without departing from the characteristics of the invention or any equivalent thereof shall be included in the scope of the invention defined in the following claims.

1. A non-polysaccharide constituent extracting from genus Dendrobium plant, the components of the non-polysaccharide constituents are shown in FIG. 2A.

2. The non-polysaccharide constituent of claim 1, wherein the genus Dendrobium plant is Dendrobium tosaense.

3. A non-polysaccharide constituent for playing as the effect drug in curing the allergic disease, the components of the non-polysaccharide constituent are shown in FIG. 2A.

4. The non-polysaccharide constituent of claim 3, wherein the non-polysaccharide constituent is extracted from a genus Dendrobium plants.

5. The non-polysaccharide constituent of claim 3, this allergic disease is related with the increase of IgE, cytokine and T_{H}2 cells.

6. The non-polysaccharide constituent of claim 3, wherein the allergic disease is atopic dermatitis.

7. A non-polysaccharide constituent having positive effect for regulating allergic diseases, the components of the non-polysaccharide constituent are shown in FIG. 2A.

8. The non-polysaccharide constituent of claim 7, wherein the non-polysaccharide constituent is extracted from a genus Dendrobium plant.

9. The non-polysaccharide constituent of claim 7, wherein the allergic disease is associated with the increase of IgE, cytokine and T_{H}2 cells.

10. The non-polysaccharide constituent of claim 7, wherein the allergic disease is atopic dermatitis.

11. A method for extracting a non-polysaccharide constituent from a genus Dendrobium plant, comprising steps of:
Step-A: taking a specific part of a genus Dendrobium plant;
Step-B: incubating the specific part taken from genus Dendrobium plants in step-A with an alcohol for obtaining a first product;
Step-C: extracting the solution containing a non-polysaccharides constituent by using at least one low-polarity solvent.

12. The method of claim 11, wherein the specific part of step-A is taken from the stem of the dry genus Dendrobium plant.

13. The method of claim 11, wherein the alcohol in step-B is methanol.

14. The method of claim 11, wherein step-C is extracting the non-polysaccharides constituent by using two solvents with different polarity from the first product prepared of step-B.

15. The method of claim 14, wherein step-C including steps of:
c′: extracting a second product by using n-hexane.
c″: obtaining a non-polysaccharides constituent of the genus Dendrobium plant from the second product by using EA.

16. The method of claim 11, wherein the genus Dendrobium plant is Dendrobium tosaense.

17. A usage of the non-polysaccharides constituent extracting by the method of claim 11 used for being an active ingredient of positive immune regulation to the allergic disease.

18. The usage of claim 17, wherein the allergic disease is related to the increase of IgE, cytokines and T_{H}2 cells.

19. The usage of claim 17, wherein the allergic disease is atopic dermatitis.
